(19) United States
(12) Patent Application Publication
(10) Pub. No.: US 2025/0367664 A1
(43) Pub. Date: Dec. 4, 2025

(54) DEVICE FOR SPERM CELL ISOLATION AND METHOD FOR SELECTION OF HIGH QUALITY SPERM CELLS

(71) Applicant: BÉEZ BIOTECH, Rennes (FR)

(72) Inventor: Shiva Kant SHUKLA, Rennes (FR)

(73) Assignee: BÉEZ BIOTECH, Rennes (FR)

(21) Appl. No.: 18/874,183

(22) PCT Filed: Jun. 13, 2023
(86) PCT No.: PCT/EP2023/065838

§ 371 Date: Dec. 12, 2024

(30) Foreign Application Priority Data
Jun. 13, 2022 (FR) 2205675

(51) Int. Cl. B01L 3/00

(52) U.S. Cl. CPC B01L 3/502761; B01L 2200/027; B01L 2200/0647; B01L 2300/1805; B01L 2400/0487

(57) ABSTRACT

A device for sperm cell selection has a support plate comprising an inlet to receive a sample of sperm cells, an outlet to collect at least some of the sperm cells comprised in the sample, and a microfluidic system therebetween. The microfluidic system comprises a basin receiving a medium and having inlet and outlet zones. The inlet and outlet provide a hydrostatic pressure difference between the zones when medium is added to the basin via the outlet, to provoke a stream of the medium from the outlet zone towards the inlet zone to initiate sperm cell migration from the inlet zone towards the outlet zone. The basin has a zone of reduced width between the zones, to form a rheotaxis zone. The sample of sperm cells undergoes a selection by passing through the rheotaxis zone during migration for collecting quality sperm cells from the outlet zone at the outlet.

The present invention is relative to a device for sperm cell selection and a method using said device to isolate quality sperm cells.

Semen analysis and quality sperm selection and isolation are essential for the practices related to “Artificial Reproductive Techniques (ARTs)”. In order to prevail over the health issues with regard to male infertility and treating oligospermia, the ARTs include “Intra Cytoplasmic Sperm Injection (ICSI)”, “Intrauterine Insemination (IUI)”, and “In-vitro Fertilization (IVF)” are being practiced.

Since the last decade, the market for ARTs has been growing. Increasing infertility rates, technological advancements, and government financial support appeared as market drivers. On the contrary, the higher cost and failure rates emerged as potential constraints. The “European Society of Human Reproduction and Embryology (ESHRE)” reported that the average possibility of pregnancy and delivery per embryo transfer is 37% and 21%, respectively (De Geyter et al., ART in Europe, 2014: Results generated from European registries by ESHRE. Hum Reprod 2018). Substandard in-vitro conditions, quality of male/female gametes, and damages related to embryos are the potential factors leading to the failures of ARTS. The screening of semen samples and subpopulation selection of quality spermatozoa (or sperm cells) is a significant step, and the efficacy of ARTs is majorly correlated with it (Oseguera-López et al., S. Novel Techniques of Sperm Selection for Improving IVF and ICSI Outcomes. Front Cell Dev Biol 2019; Pérez-Cerezales et al., The oviduct: From sperm selection to the epigenetic landscape of the embryo. Biol Reprod 2018; Sakkas et al., What can we learn from Mother Nature to improve assisted reproduction outcomes? Hum Reprod Update 2015).

Three main mechanisms rheotaxis, thermotaxis, and chemotaxis are mechanisms known to direct sperm cells towards oocytes (Giojalas and Guidobaldi, Getting to and away from the egg, an interplay between several sperm transport mechanisms and a complex oviduct physiology. Mol Cell Endocrinol 2020; Suarez, Mammalian sperm interactions with the female reproductive tract. Cell Tissue Res 2016; 363; Suarez and Pacey, Sperm transport in the female reproductive tract. Hum Reprod Update 2006).

Rheotaxis comprises the swimming and reorientation of sperm cells against a liquid flow direction. Thermotaxis comprises the migration of sperm cells induced by a temperature gradient. It is believed that thermotaxis is mainly responsible for directing the swimming of sperm cells through the follicular tube. Chemotaxis comprises the redirection of sperm cells towards oocytes and triggers sperm cell accumulation. Rheotaxis, thermotaxis, and chemotaxis are known to be occurred through the biological synergies between the swimming of sperm cells and a microenvironment hosted by the female reproductive tract. Additionally, the anatomy of the female reproductive tract encourages high-quality sperm cells migration toward the oocytes. Conclusively, the female reproductive tract facilitates the microenvironment that enables the quality selection for in-vivo conception.

Despite the known mechanism for sperm cells transportation through the female reproductive tract, reproductive health clinicians follow the ‘World Health Organization (WHO)’s protocol for the screening and quality subpopulation collection (World Health Organization, WHO laboratory manual for the examination and processing of human semen Sixth Edition. 2021). The protocol is being revised from time to time as the assessment of the minimum viability threshold ignores potentially relevant parameters such as ethnicity, environmental toxins, and the navigation capability of sperm cells in optimized conditions (Douglas et al., A novel approach to improving the reliability of manual semen analysis: A paradigm shift in the workup of infertile men. World J Mens Health 2019; Levine et al., temporal trends in sperm count: A systematic review and meta-regression analysis. Hum Reprod Update 2017; Wang and Swerdloff, Limitations of semen analysis as a test of male fertility and anticipated needs from newer tests. Fertil Steril 2014). Furthermore, the standardized protocols are manual and time-consuming as has been shown by the Applicant (Shukla et al., Automated analysis of rat sperm motility in microchannels. Biomed Phys Eng Express 2018; 4). However, WHO's manual involves the “Computer Assisted Semen Analysis (CASA)” which offers rapid and automated screening of semen samples. CASA delivers the kinematics of swimming sperm cells, though these parameters do not contemplate the microenvironment and physiological conditions of the female reproductive tract; hence, the biological significance of such parameters is still unknown. Additionally, reproductive clinicians have questioned and criticized the accuracy and the reproducibility of the CASA assay (Talarczyk-Desole et al., Manual vs. computer-assisted sperm analysis: Can CASA replace manual assessment of human semen in clinical practice? Ginekol Pol 2017; 88)

For sperm separation and isolation, WHO's manual involves standard sperm wash, density gradient centrifugation (DGC), and sperm swim-up methods, which causes DNA fragmentation in spermatozoa (Alvarez et al., Centrifugation of human spermatozoa induces sublethal damage; separation of human spermatozoa from seminal plasma by a dextran swim-up procedure without centrifugation extends their motile lifetime. Hum Reprod 1993). The prior art described the side effects of utilizing damaged male gametes in a mouse model where the substandard cells were proficient in egg fertilization; however, that leads to an alteration in gene expression and promotes a defective fetal/placental development (Fernández-Gonzalez et al., Long-term effects of mouse intracytoplasmic sperm injection with DNA-fragmented sperm on health and behavior of adult offspring. Biol Reprod 2008).

Regardless of these technological drawbacks, the CASA, DGC, and swim-up are still the most practiced protocols for sperm screening and selection, respectively. Hence, there is an enormous possibility of technological up-gradation, which can uplift the saturated success rate of ARTs.

The in-vivo mechanism of spermatozoa swimming is a complex phenomenon; hence, implementing this suggestion is not straightforward. Nonetheless, the lab-on-chip investigators exploited the advantages of microfluidic technology and established the proof-of-concept (PoC) associated with sperm swimming, semen analysis, and sperm sorting.

U.S. Pat. No. 8,535,622, US 2014/0248656 and WO 2020/041303 disclose systems for sperm cell selection.

Microfluidic engineering involves the manipulation of a small volume ranging from mL to nL. Small-volume scale and sub-millimeter channel dimension comprise a unique feature: the fluid motion in parallel streams (laminar flow), where the ratio of inertial and viscous forces is meager. This dimensionless ratio is known as Reynolds (Re) number, and it computes the predisposition of the fluid motion to develop turbulences. The laminar flow through the microchannel facilitates a high degree control, and this characteristic brings numerous advantages compared to conventional laboratory practices. The microfluidic system utilizes low sample and reagent volumes, which reduces the operational cost and improves the sensitivity and rapidness of the associated biological protocol. Microfluidic offers parallel processing, which results in high yields; moreover, technology can be integrated with external perturbation, including acoustics (Clark et al., Acoustic trapping of sperm cells from mock sexual assault samples. Forensic Sci Int Genet 2019; 41), optics (Schiffer et al., Rotational motion and rheotaxis of human sperm do not require functional CatSper channels and transmembrane Ca²⁺ signaling. EMBO J 2020), magnetic (Xu et al., agnetic Micromotors for Multiple Motile Sperm Cells Capture, Transport, and Enzymatic Release. Angew Chemie-Int Ed 2020; 59), electric (Chen, Chen, et al., Direct characterization of motion-dependent parameters of sperm in a microfluidic device: Proof of principle. Clin Chem 2013; De Wagenaar et al., Towards microfluidic sperm refinement: Impedance-based analysis and sorting of sperm cells. Lab Chip 2016; 16; De Wagenaar et al., Spermometer: electrical characterization of single boar sperm motility. Fertil Steril 2016; 106) for single-cell manipulation or separation. Microfluidic is evolving as the most practiced technique in replicating and controlling the microenvironment and in-vivo physiological conditions for human organs.

The devices of the prior art however are not satisfying, especially regarding sperm cell recovery. Devices usually have in fact low recovery properties.

Further, the execution of ARTs methods like IUI and IVF involves a certain concentration of quality spermatozoa. Although microfluidic-based approaches usually improve sperm cell quality, the throughput is still not satisfying. In other words, high throughput is still an unmet need. Nonetheless, multiplexing can be employed to increase quality cell recovery, but that increases the labor and complexity of the protocol. Furthermore, no technique from prior art combines active and passive sperm navigation mechanisms for spermatozoa quality separation.

The present invention improves the situation.

To this end, it is directed to a device for sperm cell selection having a support plate comprising an inlet to receive a sample of sperm cells, an outlet to collect at least some of the sperm cells comprised in said sample, and a microfluidic system between said inlet and outlet, wherein said microfluidic system comprises a basin arranged to receive a medium and having an inlet zone and an outlet zone respectively at the proximity of the inlet and the outlet, and wherein the inlet and the outlet are arranged to provide a hydrostatic pressure difference between said inlet zone and said outlet zone when medium is added to the basin via the outlet, so as to provoke a stream of said medium from the outlet zone towards the inlet zone to initiate sperm cell migration of sperm cells from the sample of sperm cells from the inlet zone towards the outlet zone, and wherein the inlet zone and the outlet zone are in fluidic communication with each other via a main channel arranged in the basin, the channel having a zone of reduced width arranged between the inlet zone and the outlet zone in order to form a rheotaxis zone, whereby said sample of sperm cells undergoes a selection by passing through said rheotaxis zone during said sperm cell migration thus enabling to collect a sample of quality sperm cells from the outlet zone at the outlet.

The invention is thus arranged to replicate the natural selection process of sperm cells by mimicking at least partially the female reproductive tract. In particular, the device of the invention comprises rheotaxis properties that enable to isolate quality sperm from a sperm cell sample.

The invention further proposes the device in different embodiments:
The inlet may comprise a tube having a first diameter, preferentially from 5 mm to 12 mm, and a first height, preferentially from 1.5 mm to 2.5 mm, and the outlet comprises a tube having a second diameter, preferentially from 4 mm to 5 mm, and a second height, preferentially from 3.5 mm to 5 mm (preferably from 3.5 to 4.5 mm), wherein the first diameter is greater or equal than the second diameter and the first height is less than the second height, in order to generate said hydrostatic pressure difference between the inlet zone and the outlet zone when a fluid medium is loaded. This improves the properties regarding the hydrostatic pressure difference, eventually leading to a liquid flow close to the flow of the natural female tract. This embodiment improves sperm cells selection.
The support plate may be composed of a top layer and a bottom layer, the top layer comprising said inlet and outlet, and the bottom layer comprising said basin. This improves and/or facilitates the production of the device, e.g. molding process or high-resolution SLA/DLP resin 3D printing etc.
The main channel may comprise micro-subchannels extending from the rheotaxis zone towards the outlet zone; and/or the main channel may comprise micro-subchannels extending from the inlet zone towards the rheotaxis zone. Sperm cells tend to swim adjacent to the walls of the micro-subchannels. Additionally, the microchannel confinement (from 35 μm-45 μm, more generally from 35 μm to 120 μm) elevates the progressiveness and directivity of the sperm cells. The rheotaxis zone with converging sub-microchannels enables the manipulation of the sperm cells progressivity.

In a particularly preferred embodiment of the invention, the device further comprises a rack having at least one receptacle configured to hold the support plate, the rack comprising first and second heating means, wherein said first heating means are located at proximity of the inlet and said second heating means are located at proximity of the outlet when the support plate is placed in the receptacle, the first and second heating means being respectively configured to heat at a first and a second temperature, the first temperature being lower than the second temperature so as to set a temperature gradient within the microfluidic system of the support plate in order to mimic thermotaxis.

In this preferred embodiment, the device of the invention comprises both rheotaxis and thermotaxis means. Quality sperm cell selection is drastically improved.

Another object of the invention is a method for sperm cell selection comprising the steps of:
i. providing a device as described above, having a support plate comprising an inlet, an outlet, and a microfluidic system between said inlet and outlet, the microfluidic system comprising a basin having an inlet zone and an outlet zone respectively at the proximity of the inlet and the outlet, the inlet and the outlet being arranged to provide a hydrostatic pressure difference between said inlet zone and said outlet zone when a fluid medium is added to the basin via the outlet, so as to provoke a stream of said fluid medium from the outlet zone towards the inlet zone, the inlet zone and the outlet zone being in fluidic communication with each other via a main channel arranged in the basin, wherein the channel has a zone of reduced width arranged between the inlet zone and the outlet zone in order to form a rheotaxis zone;
ii. pre-filling the basin with a fluid medium, preferentially sperm separation medium;
iii. providing a sample of sperm cells to the inlet to distribute said sample to the inlet zone of said basin;
iv. providing fluid medium to the outlet to provoke a stream of said medium from the outlet zone towards the inlet zone, thereby provoking sperm cell migration of sperm cells from the sample of sperm cells from the inlet zone towards the outlet zone;
v. selecting sperm cells from the sample of sperm cells while said sperm cells pass through said rheotaxis zone during said sperm cell migration; and
vi. collecting a sample of quality sperm cells from the outlet zone at the outlet.

The method may further comprise a step of installing temperature gradient to the support plate to mimic thermotaxis and improve said sperm cell migration. Preferentially, the basin comprises sub-micro channels. The integration of sub-microchannel is crucial as the invention not only facilitates sub-channel along the XY plane but also includes the sub-channels in the YZ plane. The invention allows the hosting of multiple sub-microchannels without compromising the quality of microfluidic-based approaches.

Other features and advantages of the invention will stand out and/or become clear upon reading the following description, which comprises specific examples given in an illustrative and non-limiting manner, as well as from the drawings in which:

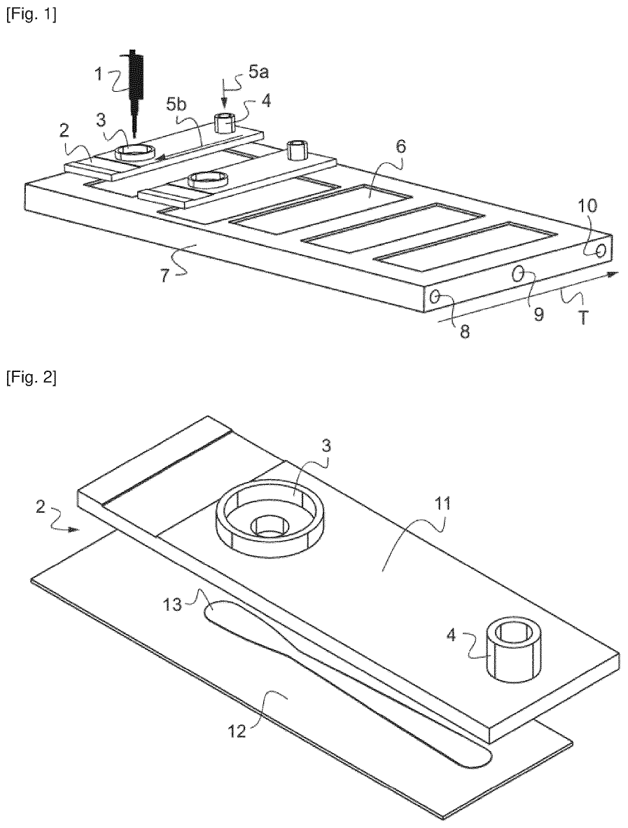

FIG. 1 shows a perspective view of an embodiment of the device according to the invention;

FIG. 2 shows a perspective view of a device according to the invention;

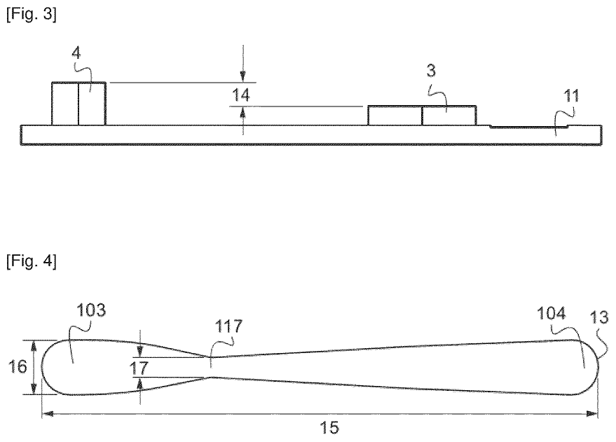

FIG. 3 shows a side view of a top layer of a device according to the invention;

FIG. 4 shows a top view of a basin of the invention;

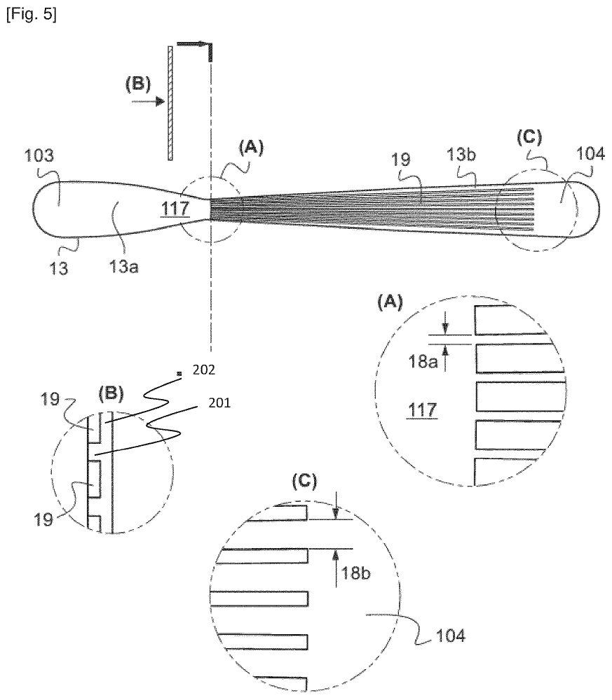

FIG. 5 shows a top view of an embodiment of a basin of the invention and further shows details of particular parts/sections of said basin;

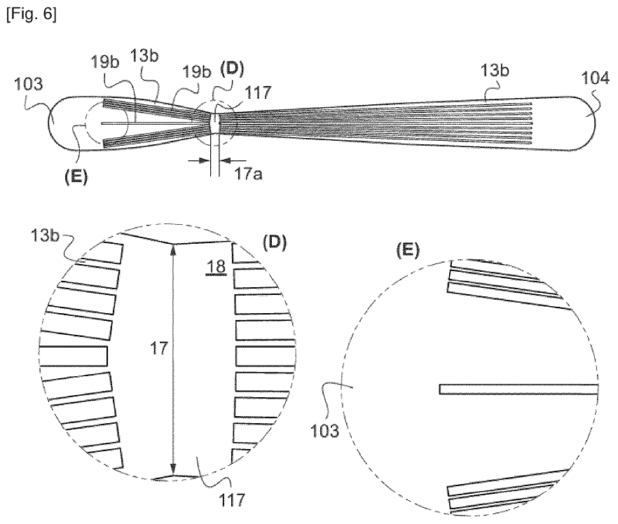

FIG. 6 shows a top view of another embodiment of a basin of the invention and further shows details of particular parts/sections of said basin;

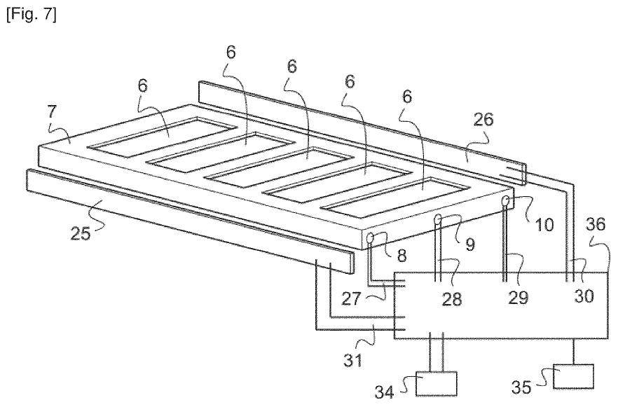

FIG. 7 shows a perspective view of a heating rack according to the invention;

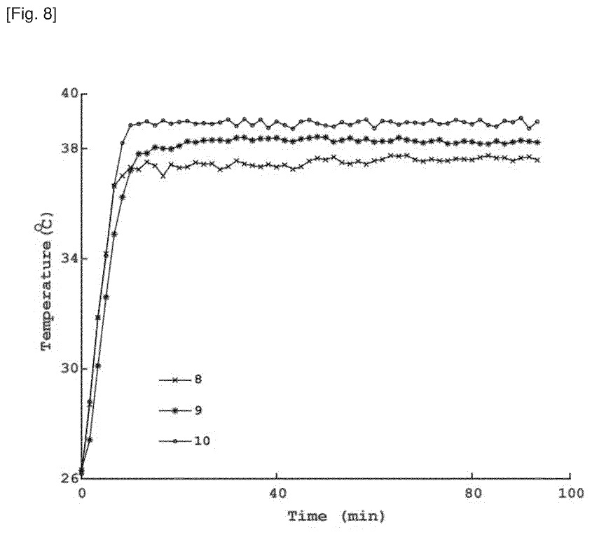

FIG. 8 shows a graph of temperatures over time in a heating rack according to the invention;

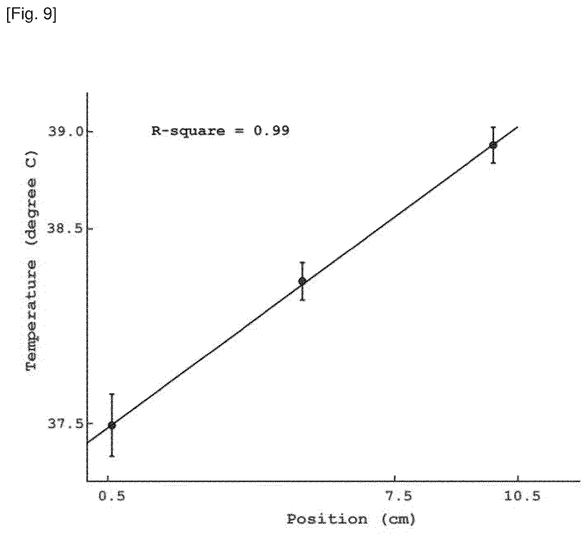

FIG. 9 shows a graph of temperatures over position in a heating rack according to the invention;

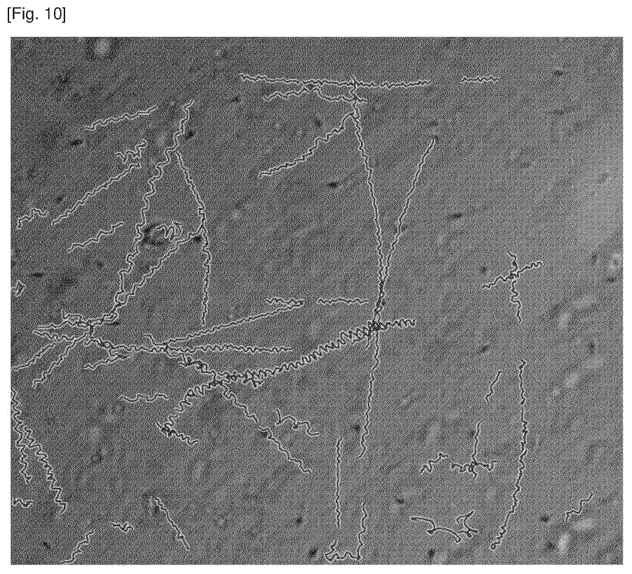

FIG. 10 shows a microscopic photograph of sperm cells and their trajectory;

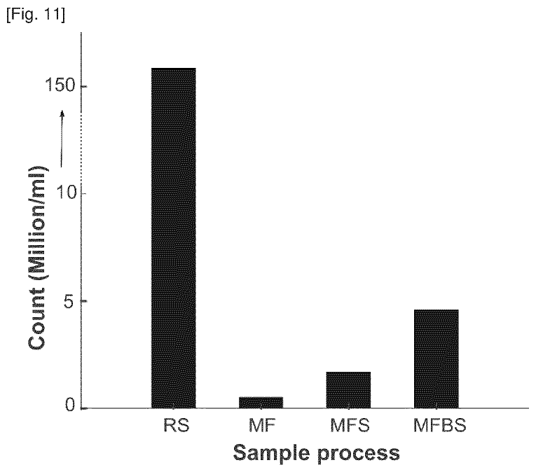

FIG. 11 shows a diagram of sperm cell concentration from a mother solution, and from processed sperm cell samples through three different embodiments of a device according to the invention;

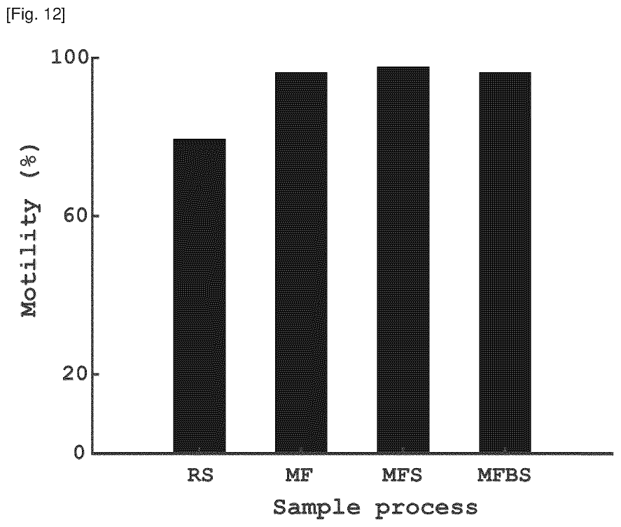

FIG. 12 shows a diagram of sperm cell motility from a mother solution, and from processed sperm cell samples through three different embodiments of a device according to the invention;

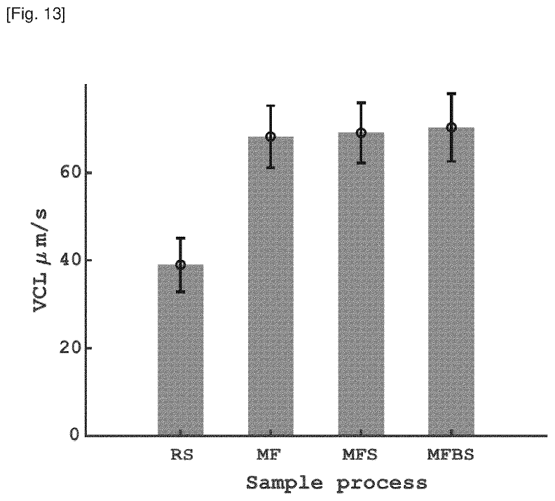

FIG. 13 shows a diagram of the instantaneous velocity of sperm cells from a mother solution, and from processed sperm cell samples through three different embodiments of a device according to the invention;

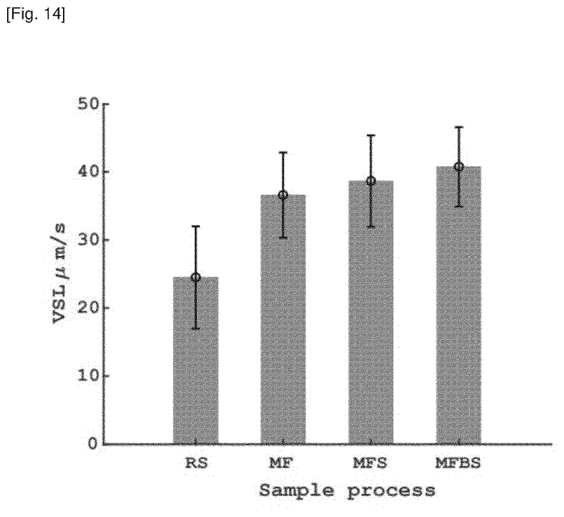

FIG. 14 shows a diagram of the progressive velocity rate of sperm cells from a mother solution, and from processed sperm cell samples through three different embodiments of a device according to the invention;

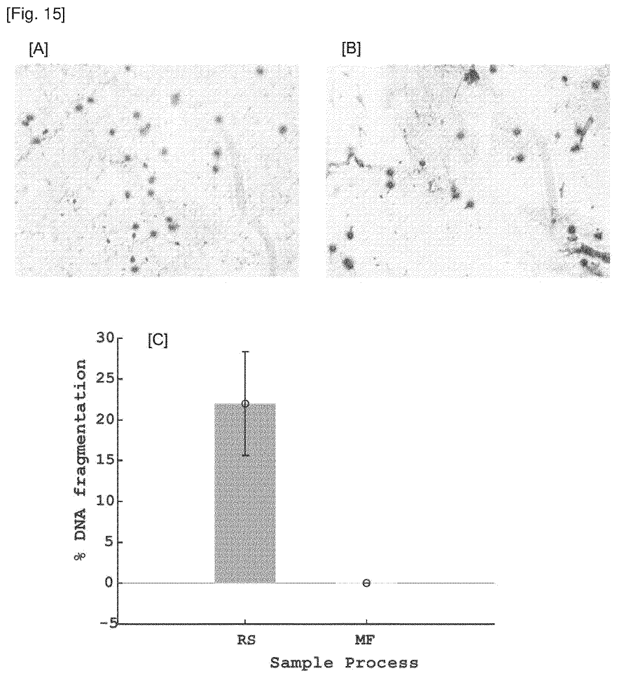

FIG. 15 shows results of a DNA fragmentation test;

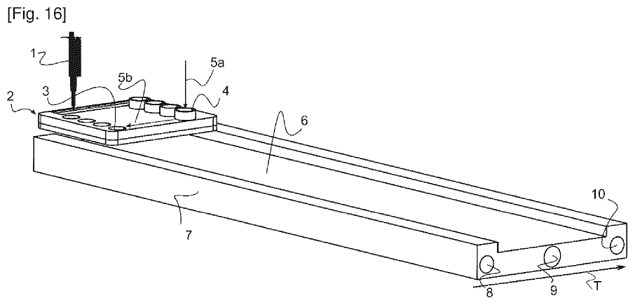

FIG. 16 shows a perspective view of an embodiment of the device according to the invention;

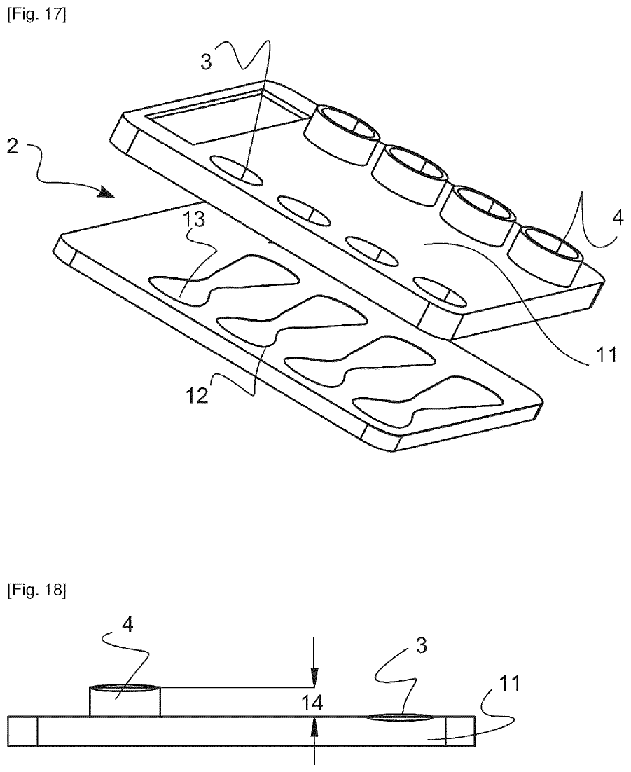

FIG. 17 shows a perspective view of a device according to the invention;

FIG. 18 shows a side view of a top layer of the device according to the invention;

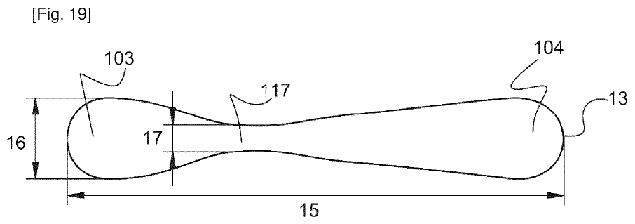

FIG. 19 shows a top view of a basin of the invention;

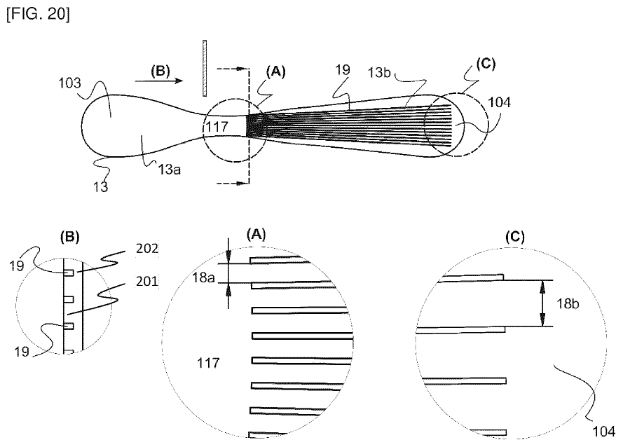

FIG. 20 shows a top view of an embodiment of a basin of the invention and further shows details of particular parts/sections of the basin;

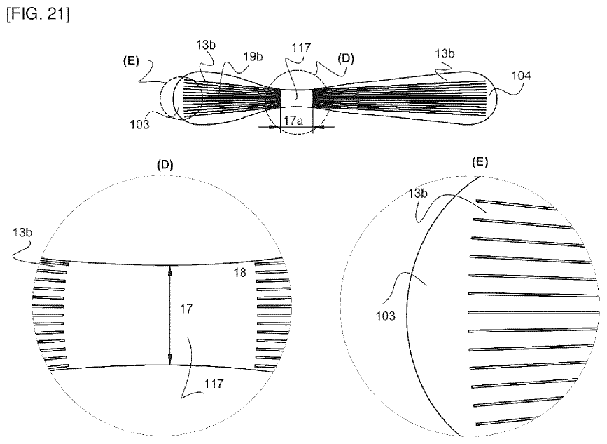

FIG. 21 shows a top view of another embodiment of a basin of the invention and further shows details of particular parts/sections of said basin;

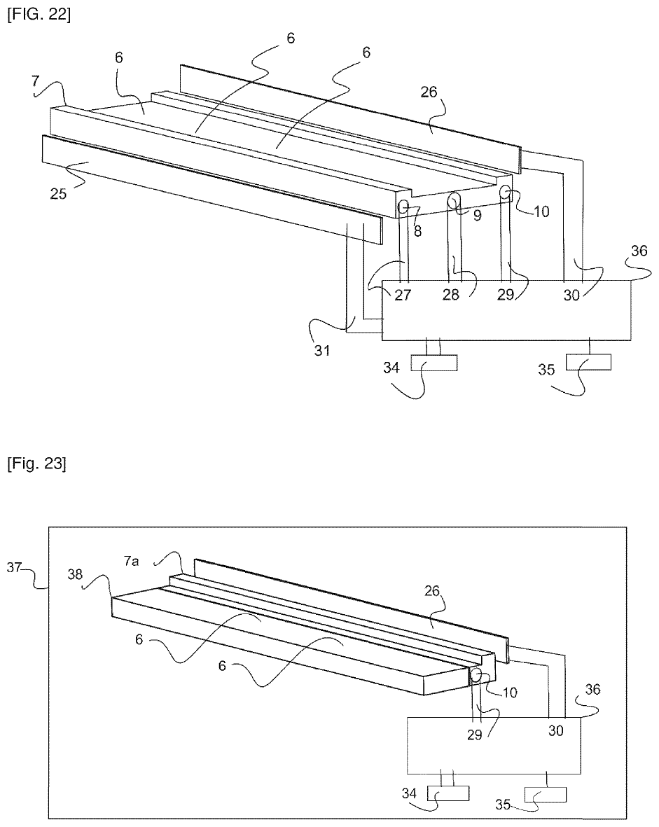

FIG. 22 shows a perspective view of a heating rack according to the invention; and

FIG. 23 shows a perspective view of a heating rack according to the invention having a single heating means.

The drawings and the description herein contain, for the most part, elements of definite nature. Therefore, description and drawings not only are being used to better understand the present invention but also to contribute to the definition therefore, when appropriate.

The term mother solution as used herein may designate a sperm cell sample. More particularly, a mother solution becomes a sperm cell sample once it is applied to the device according to the invention. The sperm cell sample is then processed through the device of the invention in order to select and/or isolate sperm cells of high quality regarding for instance motility or velocity.

The term sperm cells and the term spermatozoa are generally used to designate the same type of cells.

The present invention replicates the microenvironment of the female reproductive tract. The design of the device according to the invention exploits the fundamentals of sperm swimming mechanisms: rheotaxis, and thermotaxis. The device according to the invention enables to collect high DNA intact sperm cells subpopulation from a sample of sperm cells.

Sperm motility is triggered by the synergistic interplay of cytoskeleton and motor proteins accompanying other supplementary molecules. Any flaws in the basic structure and the functioning of these proteins weaken the motility of the cells. In order to prevail over the health issues associated with female/male infertility and treating oligospermia, the ‘Artificial Reproductive Techniques (ARTs)’ including “Intra Cytoplasmic Sperm Injection (ICSI),” “Intrauterine Insemination (IUI)”, and “In-vitro Fertilization (IVF)” have been introduced. As described above, reproductive health clinicians adhere to standard ‘World Health Organization (WHO)’s or European Association of Urology (EAU)'s protocol for the screening of semen samples and quality subpopulation separation. However, the WHO's protocol which involves sperm swim-up and density gradient centrifugation for quality sperm cell separation is not satisfying. For instance, these methods comprise centrifugation which leads to the generation of reactive oxygen species (ROS) and DNA fragmentation of sperm cells. The present invention mimics the natural selection in order to select high quality sperm cells.

Accordingly, the present invention is arranged to implement both rheotaxis and thermotaxis. As a consequence, cell migration of the spermatozoa is accelerated, resulting in high throughput (˜5×10⁶/ml) subpopulation collection with ˜100% motility and with ˜100% DNA integration. More generally, the high throughput achieves about 3% to 7% of the concentration of the cells comprised within the sample. This qualitative threshold with the yielded sub-population collection cannot be matched by any device disclosed in the prior art or by any conventional or other existing microfluidic-based system.

The device of the invention includes at least a main channel (height: approximately 80 μm to 100 μm), and preferentially micro-subchannels (height: approximately 40 μm to 50 μm). More particularly, the main channel is subjected to hydrostatic-pressure-driven flow and comprises a so-called rheotaxis zone that enables a gate-like filter system for sperm cells. The rheotaxis zone is comparable to a zone having a ventury effect. As mentioned, the device may comprise micro-subchannels. The micro-subchannels are arranged within the main channel. More particularly, the micro-subchannels may be attached to the bottom part of the main channels. The micro-subchannels assist in swimming of spermatozoa. The Applicant has surprisingly observed that the subchannel's structure accelerates the swimming of spermatozoa. This results in a higher subpopulation collection, i.e. an improved selection of sperm cells in concentration and more importantly without losing the quality properties.

The device of the invention is generally prepared by computer 3D-design followed by high-resolution 3D printing or molding techniques. The “Computer-Aided Design (CAD)” of the microfluidic circuit can be drawn in open-source FreeCAD software and further exported as an “.STL” format for injection molding or for micromanufacturing based on 3D printing. In particular, the support plate may be prepared using this technique.

According to a preferred embodiment, the device comprises a rack having a pocket or a receptacle in order to hold the support plate. The rack may comprise a gradient plate made of aluminum alloys (6800, 7075). Two polyimide tapes can be pasted in thickness-mode at both corners or opposed sides of the rack and then be heated at preferable 37° C. and 39° C., respectively. The temperatures can be controlled through the Meersttetter® 1091 thermo-electric-cooler (TEC) drivers (available from the company Meerstetter Engineering GmbH) connected with PT-100 sensors (010010TD Element 14). More generally, the invention is improved when it is actively kept at a temperature as constant as possible (i.e. constant gradient) with the help of thermal elements and independently of temperature fluctuations. To meet these requirements, the rack of the invention may use heating and/or cooling elements. The TEC Controller controls the temperature by delivering current and voltage to the thermal element, regulated by feedback from temperature sensors. In the present invention, a gradient of approximately 2° C. is achieved at the gradient plate. The gradient is diffused to the support plate (also called chip) via conduction. Temperature gradients activate the thermal receptors in sperm cells which further provoke the cell migration through the main channel and/or micro-subchannel.

The device is now described with reference to FIGS. 1 to 7.

FIG. 1 shows a perspective view of an embodiment of the device according to the invention comprising a rack 7. A support plate 2 having an elongated shape, preferably a rectangular shape, comprises an inlet 3 and an outlet 4. Both the inlet 3 and the outlet 4 have a wall of circular section and are a to apply or withdraw liquid samples such as a sperm cell sample and fluid medium respectively. The inlet 3 and the outlet 4 respectively give access to an inlet zone and an outlet zone (not shown on FIG. 1) in the interior of the support plate within a basin (also not shown on FIG. 1). The inlet zone and the outlet zone are in fluidic communication with each other. The shape and/or the configuration of the inlet 3 and the outlet 4 are chosen to provide a hydrostatic pressure difference when liquid such as a sperm cell sample is distributed to the support plate via the inlet 3 or the outlet 4 or both. Preferably, as shown in FIG. 1, the inlet 3 and the outlet 4 comprise tubes. More precisely, the inlet 3 comprises a tube having a first diameter, preferentially from 5 mm to 12 mm, and a first height, preferentially from 1.5 mm to 2.5 mm. The outlet 4 comprises a tube having a second diameter, preferentially from 4 mm to 5 mm, and a second height, preferentially from 3.5 mm to 4.5 mm or more generally from 3.5 mm to 5 mm. The first diameter is equal or greater than the second diameter and the first height is less than the second height, in order to generate a hydrostatic pressure difference between the inlet zone and the outlet zone when fluid medium is added.

The rack 7 is of elongated, preferably rectangular shape. The rack 7 comprises at least one pocket or receptacle 6 configured to hold the support plate 2. In the embodiment shown in FIG. 1 the rack 7 has five receptacles 6. This provides a ladder-like design to the rack 7. The rack 7 further comprises first heating means 8 and second heating means 10. The first heating means 8 are located at one side of the rack and the second heating means 10 are located at the opposite side of the rack. More generally, the heating means are arranged so that when the support plate 2 is placed in the receptacle 6, the first heating means 8 are located at proximity of the inlet and said second heating means 10 are located at proximity of the outlet. The first and second heating means being respectively configured to heat at a first and a second temperature, the first temperature being lower than the second temperature so as to set a temperature gradient within the microfluidic system of the support plate in order to mimic thermotaxis.

FIG. 1 generally schematizes the protocol for sperm selection with an embodiment of the device of the invention. Post prefilling of the disposable support plate 2 with a fluid medium, inlet 3 and outlet 4 are vacated with pipette 1. Subsequently, the fluid medium (˜0.065 ml or ˜0.08 ml) and sperm cell sample (˜0.05 ml or ˜0,035 ml) are injected with pipette 1 at outlet 4 and inlet 3 respectively. The height difference between outlet 4 and inlet 3 induced the flow 5 (indicated by references 5a, 5b). The sperm cells migrate against the flow, from the inlet zone towards the outlet zone, and finally reach the proximity of the outlet 4 where they can be collected. The sperm migration is also provoked by a temperature gradient T. Here, the temperature gradient T is established through a TEC card that was provided by the company Meersttetter®. The disposable support plate 2 is kept in stage pocket/receptacle 6, and heating was controlled along the disposable via polyamide tapes placed on opposite sides of the rack 7. The TEC card read and acquired the temperatures through PT-100 sensors, i.e. first heating means 8 and second heating means 10, and for validation, third heating means 9 were installed between the first and second heating means. The third heating is a negative temperature coefficient (NTC 10K) sensor.

It is now made reference to FIG. 2, which shows a perspective view of a device according to the invention and to FIG. 3, which shows a side view of a top layer of the device according to the invention. FIG. 2 shows a support plate 2. The support plate 2 comprises an upper layer plate 11 (or top layer 11) and a low layer plate 12 (or bottom layer 12). The top layer 11 comprises Ie inlet 3 and the outlet 4. The height difference 14 between the inlet 3 and the outlet 4 are shown on FIG. 3. The bottom layer 12 comprises a basin 13. The basin 13 can be filled or partially filled with fluid via the inlet 3 or via the outlet 4. More generally, both the inlet and outlet are configured to receive a fluid medium such as a sperm sample or a sperm medium. However, the stream (or flow) of the fluid according to the invention (i.e. from the outlet zone in direction of the inlet zone) is only provoked when the outlet is filled with medium. Mostly, the top layer 11 assures the fluid flow functioning via hydrostatic pressure difference between fluidic terminals, i.e. height difference 14. The bottom layer has a basin 13 assuring the fluidic design. Coupling of both layers completes the fluidic circuits, and the fluidic communication between the inlet 3 and the outlet 4.

FIG. 4 shows a top view of a basin 13 of the invention. The basin 13 is of an elongated shape. Here, it can be described as eight-shaped or as having a general aspect of a spoon. The basin 13 comprises a main channel 13a acting as microfluidic system. The basin is usually arranged to comprise a volume approximately 20 μl. More generally, depending on its length, the basin can contain a a volume of about 15 μl to 20 μl. The basin 13 further comprises an inlet zone 103 and an outlet zone 104. The inlet zone 103 is at the proximity of the inlet 3. It is filled with fluid when fluid is distributed to the inlet 3. The outlet zone 104 is at the proximity of the outlet 4. It is filled with fluid when fluid is distributed to the outlet 4. The inlet zone 103 and an outlet zone 104 are in fluidic communication with each other via the main channel 13a. As a consequence, when a fluid is distributed to either of the inlet 3 or the outlet 4, after a given time both the inlet zone 103 and an outlet zone 104 comprise the fluid. Within the microfluidic system, the basin 13 is preferably arranged within 5 mm width 16 and a 5 cm length (or 3 cm in another embodiment) 15. In this arrangement, the basin comprises a restriction of about 1.5 to 1.8 mm width 17. More generally, the aspect ratio length/wide of the basin 13 can be between 10 to 6 (or minimum 5), preferentially about 6. The ratio between the width restriction 17 and the basin mean width 16 is comprised between 0.3 and 0.36, preferentially about 0.3, additionally, the ratio between the length of rheotaxis zone 17a and the basin mean width 16 is between 0.6 and 0.8, preferentially 0.6 (not shown in the figure). The configuration of the basin 13 is so that the main channel 13a is strangled. The width of the restriction 17 is less than the width of the basin 13. More generally the inlet zone 103 and the outlet zone 104 are in fluidic communication with each other via a main channel 13a arranged in the basin 13, the channel 13a has a zone of reduced width arranged between the inlet zone 103 and the outlet zone 104 in order to form a rheotaxis zone 117. The object of the rheotaxis zone 117 is that a sample of sperm cells undergoes a selection by passing through said rheotaxis zone 117. The rheotaxis zone 117 thus acts as a gate for sperm selection. The structure of the invention only allows those sperm cells to enter another side (outlet zone) that flows upstream and are motile enough to surpass the established velocity at the rheotaxis zone 117.

FIG. 5 shows a top view of an embodiment of a basin of the invention and further shows details of particular parts/sections of the basin. The basin 13 comprises fluidic circuits wherein the main channel 13a comprises micro-subchannels 13b (also called sub-microchannels). The micro-subchannels 13b extend from the rheotaxis zone 117 towards the outlet zone 104. Zoom [A] of FIG. 5 shows the width 18a of the micro-subchannels 13b close to the rheotaxis zone 117. Zoom [C] of FIG. 5 shows the width 18b of the micro-subchannels 13b close to the outlet zone 104. In the embodiment of FIG. 5, the width 18b of the micro-subchannels close to the outlet zone is greater than the width 18a of the micro-subchannels close to the rheotaxis zone. In other words, the width of the micro-subchannels 13b is diverging from the rheotaxis zone 117 towards the outlet zone 104. The micro-subchannels 13b can be formed by placing a first set of rods 19a along the main channel 13. Zoom [A] of FIG. 5 shows detail of rods 19a placed in the main channel 13. The height of the main channel is generally comprised between 80 μm and 100 μm, preferentially about 80 μm. Accordingly, the height of the rods is generally comprised between 40 μm and 50 μm, preferentially about 40 μm (Zoom [B]). The micro-subchannels assist the sperm cell in the navigation to reach up the outlet zone 104, and eventually the outlet 4.

As mentioned above, the integration of subchannels according to the invention not only facilitates subchannels along the XY plane but also subchannels in the YZ plane. FIG. 5 (Zoom [B]) shows the so called XY-subchannels 201 and the so called YZ-subchannels 202. XY-subchannels 201 are arranged between the rods. YZ-subchannels 202 are arranged above the rods. Consequently, and as can be seen on FIG. 5 (Zoom [B]), the rods are arranged in a teeth shaped manner, this enables a combination of both XY-subchannels 201 and YZ-subchannels 202. The invention thus allows the hosting of multiple subchannels without compromising the quality of microfluidic-based approaches.

FIG. 6 shows a top view of another embodiment of a basin of the invention and further shows details of particular parts/sections of said basin. In this preferred embodiment, the mains channel 13a of the basin 13 not only comprises micro-subchannels 13b on the outlet zone 104 side (after the rheotaxis zone 117), but also comprises micro-subchannels 13b on the inlet zone 103 side (before the rheotaxis zone 117). The micro-subchannels 13b on the inlet zone 103 side are formed by placing a second set of rods 19b in the main channel 13. The rods 19b extend from the inlet zone 103 towards the rheotaxis zone 117. FIG. 6 also shows that within the rheotaxis zone 117 itself, no rods are placed. The length 17a of the rheotaxis zone 117 is usually comprised generally between 1 mm and 4 mm, preferentially about 3 mm or between 1 mm and 2.75 mm, preferentially about 2.75 mm. Zoom [D] of FIG. 6 shows the arrangement of the first set of rods 19a and the second set of rods 19b in the proximity of the rheotaxis zone 117. Zoom [E] of FIG. 6 shows the arrangement of the second set of rods 19b in the proximity of the inlet zone 103. Generally, the second set of rods 19b a converging from the inlet zone 103 towards the rheotaxis zone 117. However, at least some of the rods from the second set of rods 19b can be arranged parallelly as shown in zoom [E] of FIG. 6. More generally, FIG. 6 describes the subchannels on both sides of the rheotaxis zone. The subchannels at the inlet side assist in sperm cell propagation towards the rheotaxis zone. This arrangement facilitates the filtration of immotile sperm cells and unwanted ambiguities from the semen sample.

FIG. 7 shows a perspective view of a heating rack according to the invention. The rack 7 is similar to the one shown in FIG. 1. It is made from an approximately 1 cm thick aluminum plate and has a rectangular shape. It comprises receptacles 6 that were cut out from the aluminum plate in order to hold at least one support plate 2 of the invention. It further comprises a first polyamide heating tape 25 and a second polyamide heating tape 26 arranged on opposite length sides of the rack 7. A Meestteter® TEC board 36 was implemented to control and drive both polyamide heating tapes 25, 26. The heating modules are connected with the first 25 and second polyamide tapes. The tapes 25, 26 are respectively powered through the TEC board via a first heating connection 31 and a second heating connection 30. A first PT-100 sensor 8 and a second PT-100 sensor 10, as well as a NTC-10K sensor 9 were respectively connected to three “General Purpose Input/Output, (GPIOs)” 27, 28, 29. The coupling of the three sensors 8, 9, 10 with the TEC card 36 enables temperature reading in real-time. The “proportional-integral-derivative (PID)” functioning of the card enables the controlling of the temperature through the current flow to the resistive tapes 25, 26. The functioning of the card was handled through a computational framework 35. In working conditions of a preferred embodiment, the rack is arranged to heat the first 25 and the second 26 polyamide tapes at respectively at 37° C. and 39° C. As a consequence, the rack made of aluminum is heated at 37° C. on one length side and 39° C. on the other length side, along with the receptacles 6.

FIG. 8 and FIG. 9 show respectively a graph of temperatures over time and a graph of temperatures over position in a heating rack according to the invention. Temperature data results are shown in both figures. FIG. 8 represents the temperature measurement stability of the first PT-100 sensor 8 and a second PT-100 sensor 10, as well as an NTC-10K sensor 9. FIG. 9 shows that the temperatures at the corners (lengths sides of the rack 7) are respectively maintained at 37° C. and 39° C., and the NTC-10K sensor data show the establishment of 38° C. at the halfway point (in the middle) of the rack 7, i.e. aluminum plate. FIGS. 8, 9, show that a regular temperature gradient is installed within the rack 7. This regular temperature gradient is regularly diffused in the support plate 2 when the latter is placed in one of the receptacles 6 of the rack 7.

FIG. 10 is a microscopic image of the sperm cells. Trajectories of said sperm cells are also shown. The initial concentration of the sperm cells was assessed with a Makler chamber (Sefi medical instruments Ltd.). Motility and swimming dynamics of the sperm cells were evaluated by a computational framework, which facilitates the the trajectories of the sperm cells. The purpose of this microscopic image can be to facilitate the information associated with the data analysis (extraction of velocity and motility parameters).

### EXAMPLE

A collected sample of sperm cells (or semen sample) and sperm medium were kept at 37° C. in incubators for liquefication and preheating. The sperm medium will be incubated at 5% CO₂. At the same time, the temperature gradient is initiated within the device of the invention. The temperature is set at 37° C. and 39° C., respectively at the first and second heating means of the rack 7. The temperature gradient is installed regularly in the support plate after approximately 12 minutes. The motility and concentration of the mother solution are evaluated with a Makler chamber. Videos of moving sperm were recorded through a microscope camera (SwiftCam SC500), and image processing methods that involve background subtraction, image denoising, and intensity and region segmentation were executed for motility and sperm kinematics analysis. The device, or more precisely the basin, is prefilled (80 μl) with preheated (37° C.) sperm separation medium (Sperm Rinse™ 510312 Vitrolife). The support plate is kept at the gradient stage. After 20 minutes, 0.05 ml (or 0.35 ml in another example) of the liquified semen sample is loaded at the device's inlet, and immediately, 80 μl (or 65 μl in another example) of the sperm medium is added at the outlet. The rheotaxis and thermotaxis-based sperm migration occur and after ˜45 minutes (or even after 30 minutes), approximately 80 μl (or 65 μl in another example) of sperm sample is collected from the outlet. The motility of the subpopulation collection is also completed with a Makler chamber and microscope (Amscope T720Q). An object tracking algorithm module based on the Kalman filter and Hungarian algorithm was implemented to extract the XY trajectories from the images.

FIGS. 11, 12, 13, and 14 show measurements and results of the above example carried out in three different embodiments of the invention.

FIGS. 11, 12, 13, and 14 show measurements and results of the above example carried out in the devices of the invention shown in embodiments of FIGS. 4, 5, and 6. RS represents data from the raw sperm sample (mother solution). MF represents data from sperm cells selected and collected at the outlet from the device of the invention shown in FIG. 4, i.e. having a main channel without micro-subchannels. MFS represents data from sperm cells selected and collected at the outlet from the device of the invention shown in FIG. 5, i.e. having a main channel and micro-subchannels on the outlet side. MFBS represents data from sperm cells selected and collected at the outlet from the device of the invention shown in FIG. 6, i.e. having a main channel and micro-subchannels on the outlet side and on the inlet side.

FIG. 11 shows sperm cell concentration outcomes. FIG. 12 shows the motility of sperm cells. Nearly ˜100% of the motile cells were separated and isolated from the initial sperm sample. FIG. 13 shows the instantaneous velocity of the sperm cells improved in the collected subpopulation of quality sperm isolated from the initial sperm sample. FIG. 14 shows that the progressive rate of sperm cells has been improved.

FIG. 15 represents the outcomes of the DNA fragmentation test. [A] and [B] are the representative microscopic images (1280×940 px² with 2.48 px/μm) for the mother solution (RS) and the microfluidic embodiment (MF). The Halosperm® G2 DNA fragmentation kit and protocol were followed to evaluate the DNA fragmentation (provided by the Halotech®). The big-halo head represents no-fragmentation or degradation, on the other hand small-halo head or no-halo shows the fragmentation and degradation. The figure [C] validates that sperm selection executed with embodiment 13 facilitates the sperm subpopulation with ˜100% DNA integrity.

Other arrangements have been carried out. According to an embodiment, the temperatures set by the heating means of the rack 7 are comprised between 36° C. and 40° C. In particular, the first temperature is set between 36° C. and 38° C., preferentially 37° C., and the second temperature is set between 38° C. and 40° C., preferentially 39° C.

In a more general sense, the object of the invention is a device for sperm cell selection having means to mimic rheotaxis and means to mimic for thermotaxis.

Another object of the invention is a Kit for sperm cell selection comprising:
A. a device having a support plate comprising an inlet to receive a sample of sperm cells, an outlet to collect at least some of the sperm cells comprised in said sample, and a microfluidic system between said inlet and outlet,
wherein said microfluidic system comprises a basin arranged to receive a medium and having an inlet zone and an outlet zone respectively at the proximity of the inlet and the outlet,
and wherein the inlet and the outlet are arranged to provide a hydrostatic pressure difference between said inlet zone and said outlet zone when medium is added to the basin via the outlet, so as to provoke a stream of said medium from the outlet zone towards the inlet zone to initiate sperm cell migration of sperm cells from the sample of sperm cells from the inlet zone towards the outlet zone,
and wherein the inlet zone and the outlet zone are in fluidic communication with each other via a main channel arranged in the basin, the channel having a zone of reduced width arranged between the inlet zone and the outlet zone in order to form a rheotaxis zone,
whereby said sample of sperm cells undergoes a selection by passing through said rheotaxis zone during said sperm cell migration thus enabling to collect a sample of quality sperm cells from the outlet zone at the outlet; and
B. a rack having at least one receptacle configured to hold the support plate, the rack comprising first and second heating means, wherein said first heating means are located at proximity of the inlet and said second heating means are located at proximity of the outlet when the support plate is placed in the receptacle, the first and second heating means being respectively configured to heat at a first and a second temperature, the first temperature being lower than the second temperature so as to set a temperature gradient within the microfluidic system of the support plate in order to mimic thermotaxis.

The Applicant has further developed embodiments, close to those described above, that however show particular good results.

FIG. 16 shows a perspective view of an embodiment of the device according to the invention comprising a rack 7. A support plate 2 having an elongated rectangular shape, that comprises an inlet 3 and an outlet 4. Both the inlet 3 and the outlet 4 have a wall of circular section and are arranged to apply or withdraw liquid samples such as a sperm cell sample and fluid medium respectively. The inlet 3 and the outlet 4 respectively give access to an inlet zone and an outlet zone (not shown) in the interior of the support plate within a basin (not shown). The inlet zone and the outlet zone are in fluidic communication with each other. The shape and/or the configuration of the inlet 3 and the outlet 4 are chosen to provide a hydrostatic pressure difference when liquid such as a sperm cell sample is distributed to the support plate via the inlet 3 or the outlet 4 or both. Preferably, as shown in FIG. 16, the inlet 3 and the outlet 4 comprise tubes. More precisely, the inlet 3 comprises a tube having a first diameter, preferentially from 5 mm to 12 mm, and a first height, preferentially from 1.5 mm to 2.5 mm. The outlet 4 comprises a tube having a second diameter, preferentially from 4 mm to 5 mm, and a second height, preferentially from 3.5 mm to 4.5 mm or more generally from 3.5 mm to 5 mm. The first diameter is equal or greater than the second diameter and the first height is less than the second height, in order to generate a hydrostatic pressure difference between the inlet zone and the outlet zone when fluid medium is added.

The rack 7 is elongated, preferably in rectangular shape. The rack 7 comprises at least one pocket or receptacle 6 configured to hold the support plate 2. In the embodiment shown in FIG. 16 the rack 7 has continuous receptacles 6 which can host multiple support plates 2. This provides a ladder-like design to the rack 7. The rack 7 further comprises first heating means 8 and second heating means 10. The first heating means 8 are located at one side of the rack and the second heating means 10 are located at the opposite side of the rack. More generally, the heating means are arranged so that when the support plate 2 is placed in the receptacle 6, the first heating means 8 are located at proximity of the inlet and said second heating means 10 are located at proximity of the outlet. The first and second heating means being respectively configured to heat at a first and a second temperature, the first temperature being lower than the second temperature so as to set a temperature gradient within the microfluidic system of the support plate in order to mimic thermotaxis.

FIG. 16 schematizes the protocol for sperm selection with an embodiment of the device of the invention. Post prefilling of the disposable support plate 2 with a fluid medium, inlet 3 and outlet 4 are vacated with pipette 1. Subsequently, the fluid medium (˜0.065 ml or ˜0.08 ml) and sperm cell sample (˜0.05 ml or ˜0,035 ml) are injected with a pipette 1 (˜0.05 ml or ˜0,035 ml) at the outlet 4 and at the inlet 3 respectively. The height difference between outlet 4 and inlet 3 induced the flow 5 (indicated by references 5a, 5b). The sperm cells migrate against the flow, from the inlet zone towards the outlet zone, and finally reach the proximity of outlet 4 where they can be collected. The sperm migration is also provoked by a temperature gradient T. Here, the temperature gradient T is established through a TEC card that was provided by the company Meersttetter®. The disposable support plate 2 is kept in stage pocket/receptacle 6, and heating was controlled along the disposable via polyamide tapes placed on opposite sides of the rack 7. The TEC card read and acquired the temperatures through PT-100 sensors, i.e. first heating means 8 and second heating means 10, and for validation, third heating means 9 were installed between the first and second heating means. The third heating is a negative temperature coefficient (NTC 10K) sensor.

FIG. 17 shows a perspective view of a device according to the invention, and FIG. 18 shows a side view of a top layer of the device according to the invention. FIG. 17 shows a support plate 2. The support plate 2 comprises an upper layer plate 11 (or top layer 11) and a low layer plate 12 (or bottom layer 12). The top layer 11 comprises Ie inlet 3 and the outlet 4. The height difference 14 between the inlet 3 and the outlet 4 are shown on FIG. 18. The bottom layer 12 comprises a basin 13. The basin 13 can be filled or partially filled with fluid via the inlet 3 or via the outlet 4. More generally, both the inlet and outlet are configured to receive a fluid medium such as a sperm sample or a sperm medium. However, the stream (or flow) of the fluid according to the invention (i.e. from the outlet zone in the direction of the inlet zone) is only provoked when the outlet is filled with medium. Mostly, the top layer 11 assures the fluid flow functioning via hydrostatic pressure difference between fluidic terminals, i.e. height difference 14. The bottom layer has a basin 13 assuring the fluidic design. Coupling of both layers completes the fluidic circuits, and the fluidic communication between the inlet 3 and the outlet 4.

FIG. 19 shows a top view of a basin 13 of the invention. The basin 13 is of an elongated shape. Here, it can be described as eight-shaped or as having a general aspect of a spoon. The basin 13 comprises a main channel 13a acting as microfluidic system. The basin is usually arranged to comprise a volume approximately 20 μl. More generally, depending on its length, the basin can contain a a volume of about 15 μl to 20 μl. The basin 13 further comprises an inlet zone 103 and an outlet zone 104. The inlet zone 103 is at the proximity of the inlet 3. It is filled with fluid when fluid is distributed to the inlet 3. The outlet zone 104 is at the proximity of the outlet 4. It is filled with fluid when fluid is distributed to the outlet 4. The inlet zone 103 and an outlet zone 104 are in fluidic communication with each other via the main channel 13a. As a consequence, when a fluid is distributed to either of the inlet 3 or the outlet 4, after a given time both the inlet zone 103 and an outlet zone 104 comprise the fluid. Within the microfluidic system, the basin 13 is preferably arranged within 5 mm width 16 and a 5 cm length (or 3 cm in another embodiment) 15. In this arrangement, the basin comprises a restriction of about 1.5 to 1.8 mm width 17. More generally, the aspect ratio length/wide of the basin 13 can be between 10 to 6 (or minimum 5), preferentially about 6. The ratio between the width restriction 17 and the basin mean width 16 is comprised between 0.3 and 0.36, preferentially about 0.3, additionally, the ratio between the length of rheotaxis zone 17a and the basin mean width 16 is between 0.6 and 0.8, preferentially 0.6 (not shown in figure). The configuration of the basin 13 is so that the main channel 13a is strangled. The width of the restriction 17 is less than the width of the basin 13. More generally the inlet zone 103 and the outlet zone 104 are in fluidic communication with each other via a main channel 13a arranged in the basin 13, the channel 13a has a zone of reduced width arranged between the inlet zone 103 and the outlet zone 104 in order to form a rheotaxis zone 117. The object of the rheotaxis zone 117 is that a sample of sperm cells undergoes a selection by passing through said rheotaxis zone 117. The rheotaxis zone 117 thus acts as a gate for sperm selection. The structure of the invention only allows those sperm cells to enter another side (outlet zone) that flows upstream and are motile enough to surpass the established velocity at the rheotaxis zone 117.

FIG. 20 shows a top view of an embodiment of a basin of the invention and further shows details of particular parts/sections of the basin. The basin 13 comprises fluidic circuits wherein the main channel 13a comprises micro-subchannels 13b (also called sub-microchannels). The micro-subchannels 13b extend from the rheotaxis zone 117 towards the outlet zone 104. Zoom [A] of FIG. 20 shows the width 18a of the micro-subchannels 13b close to the rheotaxis zone 117. Zoom [C] of FIG. 20 shows the width 18b of the micro-subchannels 13b close to the outlet zone 104. In the embodiment of FIG. 20, the width 18b of the micro-subchannels close to the outlet zone is greater than the width 18a of the micro-subchannels close to the rheotaxis zone. In other words, the width of the micro-subchannels 13b is diverging from the rheotaxis zone 117 towards the outlet zone 104. The micro-subchannels 13b can be formed by placing a first set of rods 19a along the main channel 13. Zoom [A] of FIG. 20 shows detail of rods 19a placed in the main channel 13. The height of the main channel is generally comprised between 80 μm and 100 μm, preferentially about 80 μm. Accordingly, the height of the rods is generally comprised between 40 μm and 50 μm, preferentially about 40 μm (Zoom [B]). The micro-subchannels assist the sperm cell in the navigation to reach up the outlet zone 104, and eventually the outlet 4.

As mentioned above, the integration of micro-subchannels according to the invention not only facilitates channels along the XY plane but also channels in the YZ plane. FIG. 20 (Zoom [B]) shows the so called XY-micro-subchannels 201 and the so called YZ-micro-subchannels 202. XY-micro-subchannels 201 are arranged between the rods. YZ-micro-subchannels 202 are arranged above the rods. Consequently, and as can be seen on FIG. 20 (Zoom [B]), the rods are arranged in a teeth shaped manner, this enables a combination of both XY-subchannels 201 and YZ-subchannels 202. The invention thus allows the hosting of multiple subchannels without compromising the quality of microfluidic-based approaches.

FIG. 21 shows a top view of another embodiment of a basin of the invention and further shows details of particular parts/sections of said basin. In this preferred embodiment, the mains channel 13a of the basin 13 not only comprises micro-subchannels 13b on the outlet zone 104 side (after the rheotaxis zone 117), but also comprises micro-subchannels 13b on the inlet zone 103 side (before the rheotaxis zone 117). The micro-subchannels 13b on the inlet zone 103 side are formed by placing a second set of rods 19b in the main channel 13. The rods 19b extend from the inlet zone 103 towards the rheotaxis zone 117. FIG. 21 also shows that within the rheotaxis zone 117 itself, no rods are placed. The length 17a of the rheotaxis zone 117 is usually comprised generally between 1 mm and 4 mm, preferentially about 3 mm or between 1 mm and 2.75 mm, preferentially about 2.75 mm. Zoom [D] of FIG. 21 shows the arrangement of the first set of rods 19a and the second set of rods 19b in the proximity of the rheotaxis zone 117. Zoom [E] of FIG. 21 shows the arrangement of the second set of rods 19b in the proximity of the inlet zone 103. Generally, the second set of rods 19b converges from the inlet zone 103 towards the rheotaxis zone 117. However, at least some of the rods from the second set of rods 19b can be arranged parallelly as shown in zoom [E] of FIG. 21. More generally, FIG. 21 describes the subchannels on both sides of the rheotaxis zone. The subchannels at the inlet side assist in sperm cell propagation towards the rheotaxis zone. This arrangement facilitates the filtration of immotile sperm cells and unwanted ambiguities from the semen sample.

FIG. 22 shows a perspective view of a heating rack according to the invention. The rack 7 is similar to the one shown in FIG. 16. It is made from an approximately 1 cm thick aluminum plate and has a rectangular shape. It comprises receptacles 6 that were cut out from the aluminum plate in order to hold at least one support plate 2 of the invention. It further comprises a first polyamide heating tape 25 and a second polyamide heating tape 26 arranged on opposite length sides of the rack 7. A Meestteter® TEC board 36 was implemented to control and drive both polyamide heating tapes 25, 26. The heating modules are connected with the first 25 and second polyamide tapes. The tapes 25, 26 are respectively powered through the TEC board via a first heating connection 31 and a second heating connection 30. A first PT-100 sensor 8 and a second PT-100 sensor 10, as well as a NTC-10K sensor 9 were respectively connected to three “General Purpose Input/Output, (GPIOs)” 27, 28, 29. The coupling of the three sensors 8, 9, 10 with the TEC card 36 enables temperature reading in real-time. The “proportional-integral-derivative (PID)” functioning of the card enables the controlling of the temperature through the current flow to the resistive tapes 25, 26. The functioning of the card was handled through a computational framework 35. In working conditions of a preferred embodiment, the rack is arranged to heat the first 25 and the second 26 polyamide tapes at respectively at 37° C. and 39° C. As a consequence, the rack made of aluminum is heated at 37° C. on one length side and 39° C. on the other length side, along with the receptacles 6.

In a specific embodiment, thermotaxis can be achieved by integrating only one unit of the heating source where the heating stage will be placed inside the incubator at an inferior temperature and the heating stage at a comparatively higher temperature.

Accordingly, the invention further provides an embodiment wherein the device comprises a rack (7) having at least one receptacle (6) configured to hold the support plate (2), the rack comprising a single heating means, wherein said single heating means is located at proximity of the inlet (3) or is located at proximity of the outlet (4) when the support plate (2) is placed in the receptacle (7). The single heating means is configured to heat at a selected temperature which is higher than room temperature. A preferred temperature is 39° C. Surprisingly, this specific embodiment prohibits bubble formation within the medium. Thus, the results of this embodiment are particularly satisfying. The selected temperature installs a particular gradient within the microfluidic system of the support plate and thus effectively mimics thermotaxis.

FIG. 23 shows an alternative configuration for generating thermotaxis. This configuration involves an aluminum metal bar 7a comprising receptacle 6 (or pocket) and attached to a polyamide heating ribbon 26. The metal bar 7a is attached with an adjacent non-conductive board 38 that accomplishes the receptacle 6 (or hosting pocket) for the support plate (sometimes called fluidic unit). A TEC Meerstetter® 36 printed circuit board is implemented to control and drive the polyamide heating ribbon 26. The aluminum metal bar 7a contains a PT 100 sensor 10, where data acquisition occurs via (GPIO) 29. Coupling the sensor 10 to the TEC board 36 enables real-time temperature readout and control. The card's operation has been managed according to configuration 35, previously described in connection with FIG. 7. Here, the second temperature is activated by an incubator 37.

The device of the invention is a pioneer in the field of biomimetics.

1. Device for sperm cell selection having a support plate comprising an inlet to receive a sample of sperm cells, an outlet to collect at least some of the sperm cells comprised in said sample, and a microfluidic system between said inlet and outlet,
wherein said microfluidic system comprises a basin arranged to receive a medium and having an inlet zone and an outlet zone respectively at the proximity of the inlet and the outlet,
wherein the inlet and the outlet are arranged to provide a hydrostatic pressure difference between said inlet zone and said outlet zone when medium is added to the basin via the outlet, so as to provoke a stream of said medium from the outlet zone towards the inlet zone to initiate sperm cell migration of sperm cells from the sample of sperm cells from the inlet zone towards the outlet zone,
wherein the inlet zone and the outlet zone are in fluidic communication with each other via a main channel arranged in the basin, the channel having a zone of reduced width arranged between the inlet zone and the outlet zone in order to form a rheotaxis zone,
wherein the main channel comprises micro-subchannels extending from the rheotaxis zone towards the outlet zone, and further comprises micro-subchannels extending from the inlet zone towards the rheotaxis zone,
the device being configured such that said sample of sperm cells undergoes a selection by passing through said rheotaxis zone during said sperm cell migration thus enabling to collect a sample of quality sperm cells from the outlet zone at the outlet.

2. The device according to claim 1, wherein the inlet comprises a tube having a first diameter and a first height, and the outlet comprises a tube having a second diameter and a second height, wherein the first diameter is greater or equal than the second diameter and the first height is less than the second height, in order to generate said hydrostatic pressure difference between the inlet zone and the outlet zone when medium is added.

3. The device according to claim 1, wherein the support plate is composed of a top layer and a bottom layer, the top layer comprising said inlet and outlet, and the bottom layer comprising said basin.

4. The device according to claim 1, wherein at least some of the micro-subchannels are converging towards the rheotaxis zone.

5. The device according to claim 1, wherein the main channel comprises rods arranged along said basin that are extending from the inlet zone towards the rheotaxis zone and/or from the rheotaxis zone towards the outlet zone, and wherein said micro-subchannels are arranged between and above said rods.

6. The device according to claim 1, wherein said micro-subchannels are arranged in both the XY plane and the YZ plane of the basin.

7. The device according to claim 1, wherein the device further comprises a rack having at least one receptacle configured to hold the support plate, the rack comprising first and second heating means, wherein said first heating means are located at proximity of the inlet and said second heating means are located at proximity of the outlet when the support plate is placed in the receptacle, the first and second heating means being respectively configured to heat at a first and a second temperature, the first temperature being lower than the second temperature so as to set a temperature gradient within the microfluidic system of the support plate in order to mimic thermotaxis.

8. Method for sperm cell selection comprising the steps of:
i. providing a device according to claim 1;
ii. pre-filling the basin with fluid medium;
iii. providing a sample of sperm cells to the inlet to distribute said sample to the inlet zone of said basin;
iv. providing fluid medium to the outlet to provoke a stream of said medium from the outlet zone towards the inlet zone, thereby provoking sperm cell migration of sperm cells from the sample of sperm cells from the inlet zone towards the outlet zone;
v. selecting sperm cells from the sample of sperm cells while said sperm cells pass through said rheotaxis zone during said sperm cell migration; and
vi. collecting a sample of quality sperm cells from the outlet zone at the outlet.

9. The method according to claim 8, further comprising the step of installing a temperature gradient to the support plate to mimic thermotaxis and improve said sperm cell migration.

10. The method according to claim 8, wherein the fluid medium of step ii. is a sperm separation medium.

11. The device according to claim 2, wherein the first diameter is within a range from 5 mm to 12 mm.

12. The device according to claim 2, wherein the first height is within a range from 1.5 mm to 2.5 mm.

13. The device according to claim 2, wherein the second diameter is within a range from 4 mm to 5 mm.

14. The device according to claim 2, wherein the second height is within a range from 3.5 mm to 4.5 mm.
